(19) United States
(12) Patent Application Publication
(10) Pub. No.: US 2017/0043116 A1
(43) Pub. Date: Feb. 16, 2017

(54) MEDICAL APPARATUS AND PROGRAM

(71) Applicant: Seiko Epson Corporation, Tokyo (JP)

(72) Inventors: Hideki TANAKA, Chino (JP); Yuya MARUYAMA, Chino (JP); Akihiko YAMAMOTO, Yamagata (JP); Masahiko KOBAYASHI, Chino (JP)

(21) Appl. No.: 15/235,486

(22) Filed: Aug. 12, 2016

(30) Foreign Application Priority Data
Aug. 13, 2015 (JP) 2015-159743

(51) Int. Cl. A61M 21/00

(52) U.S. Cl. CPC A61M 21/00; A61M 2021/005

(57) ABSTRACT

A moving image in which a therapy target part repeatedly moves is displayed on a screen member along with a black background.

### BACKGROUND

1. Technical Field

The present invention relates to a medical apparatus.

2. Related Art

There is a therapy in which a patient is made to visually recognize moving images in which a lost part moves and also to imagine that the lost part moves in accordance with the moving images, in order to alleviate pain (phantom limb pain) of a lost limb. In the above-described way, it is considered that the conclusion that “a phantom limb is not present” in the brain of the patient is induced, and thus the pain is alleviated. JP-A-2004-298430 discloses a medical apparatus used for the therapy.

The therapy focuses on generation of an illusion in the brain of the patient by interlocking the visual recognition with imagination. The therapy may also be effective in pain other than a phantom limb pain, or rehabilitation of quadriplegia.

### SUMMARY

An advantage of some aspects of the invention is to improve a therapeutic effect by generating a stronger illusion on the basis of the related art.

The advantage can be achieved by the following configurations.

According to one aspect of the invention, a medical apparatus is provided. The medical apparatus displays a moving image in which a therapy target part repeatedly moves on a screen member along with a black background. According to the aspect, a background color is black, and thus an immersive feeling is enhanced. As a result, a stronger illusion is generated, and thus a therapeutic effect is improved.

In the aspect, at least a background portion may be black in the screen member. According to the aspect, it is possible to easily realize a black background.

In the aspect, the moving image maybe projected onto the screen member along with the black background. According to this aspect, it is possible to easily realize a black background. For example, only the therapy target part may not be projected.

In the aspect, the screen member may be disposed between a specific part which is at least one of the therapy target part and a counter part of the therapy target part, and the eyes of a patient. According to the aspect, a moving image can be visually recognized, and the specific part can be made to come close to a position at which the moving image is displayed. As a result, a stronger illusion is generated, and thus a therapeutic effect is improved.

In the aspect, the screen member may be translucent. According to the aspect, the specific part can be visually recognized along with the moving image. As a result, a stronger illusion is generated, and thus a therapeutic effect is improved.

In the aspect, the screen member may have a shape rising up along the specific part during display of the moving image, and the moving image may be displayed on the rising-up part. According to the aspect, a stronger illusion is generated, and thus a therapeutic effect is improved.

The invention may be implemented in various aspects other than the above-described aspects. For example, the invention may be implemented in aspects such as a therapy method or a program for performing the therapy method.

### BRIEF DESCRIPTION OF THE DRAWINGS

The invention will be described with reference to the accompanying drawings, wherein like numbers reference like elements.

FIG. 1 is a side view of a medical apparatus.

FIG. 2 is a perspective view illustrating a cheesecloth, a transparent plate, and a box.

FIG. 3 is a diagram illustrating a state in which a patient puts the right hand thereof into the box.

FIG. 4 is a block diagram illustrating an internal configuration of a projector.

FIG. 5 is a flowchart illustrating a projection process.

FIG. 6 is a diagram illustrating an initial image.

FIG. 7 is an enlarged view of a dialogue screen.

FIG. 8 is a diagram illustrating a first state during reproduction of moving images.

FIG. 9 is a diagram illustrating a second state during reproduction of moving images.

FIG. 10 is a side view of a medical apparatus in Embodiment 2.

FIG. 11 is a diagram illustrating a state in which a patient puts both hands thereof between a cloth and a stand.

FIG. 12 is a diagram illustrating a first state during reproduction of moving images.

FIG. 13 is a sectional view in the first state.

FIG. 14 is a diagram illustrating a second state during reproduction of moving images.

FIG. 15 is a sectional view in the second state.

### DESCRIPTION OF EXEMPLARY EMBODIMENTS

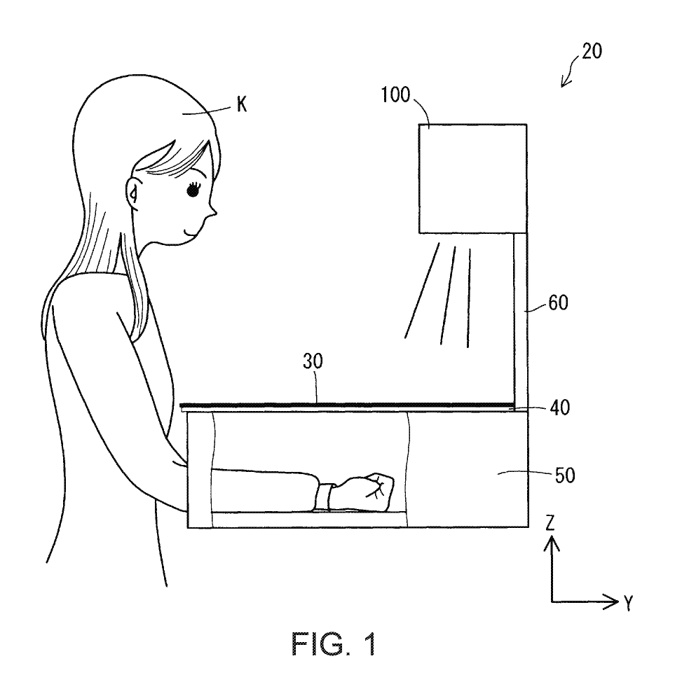

Embodiment 1 will be described. FIG. 1 is a side view of a medical apparatus 20. The medical apparatus 20 is used for therapy of phantom limb pain or rehabilitation of functions of the fingers. The medical apparatus 20 includes a cheesecloth 30, a transparent plate 40, a box 50, a column 60, and a projector 100. FIG. 1 illustrates a part of the box 50 which is virtually notched. In the present embodiment and Embodiment 2 which will be described later, a vertical direction is set as a Z direction, and a horizontal plane is set as an XY plane.

The projector 100 is a display device which projects a therapeutic moving image onto the cheesecloth 30 as will be described later in detail. The column 60 is fixed to the box 50 and supports the projector 100.

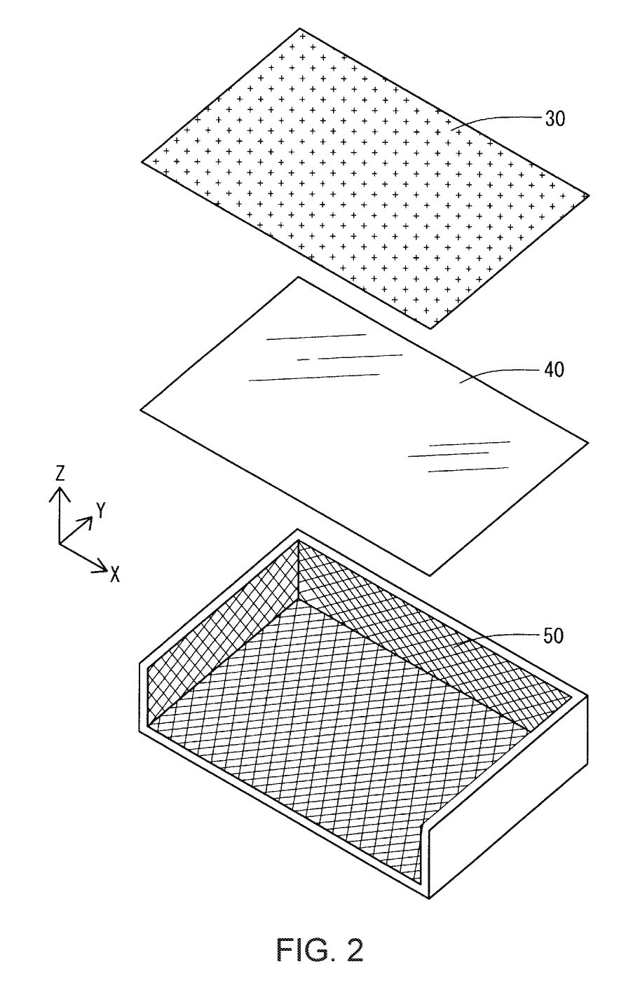

FIG. 2 is a perspective view illustrating the cheesecloth 30, the transparent plate 40, and the box 50. During use of the medical apparatus 20, as illustrated in FIG. 1, the transparent plate 40 is placed on the box 50, and the cheesecloth 30 is placed thereon.

The cheesecloth 30 is a cloth which is roughly and plainly woven, and an opposite side is viewed through the texture. Threads forming the cheesecloth 30 are black. The cheesecloth 30 functions as a screen member which displays an image projected by the projector 100.

Here, black in the present embodiment is defined. Black in the present embodiment satisfies at least one of the following two conditions: (1) Optical density OD>1.5, and (2) in the L^{*}a^{*}b^{*} system, lightness L^{*}<18, a^{*}<9, and b^{*}<20.

The transparent plate 40 is a plate made of acryl, glass, or the like, and is a member which is colorless and transparent, and is rigid. The transparent plate 40 is placed on the box 50 in order to spread out the cheesecloth 30.

The box 50 has a substantially rectangular parallelepiped shape. One of four side faces of the box 50 is not provided so that a patient K can place the hands and the forearms thereof on the bottom of the box. In the present embodiment, the hand indicates the front part from the wrist. The box 50 does not have a top face so that the patient K can visually recognize the hands put into the box. Inner surfaces of the bottom and the three side faces of the box 50 are black.

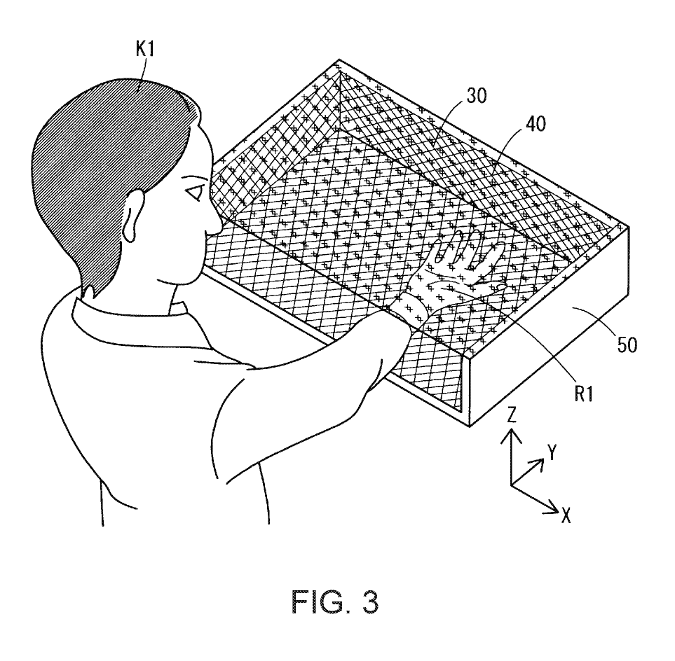

FIG. 3 illustrates a state in which a patient K1 puts the right hand R1 into the box 50. The patient K1 has lost the left arm thereof, and thus feels phantom limb pain of the left arm. A therapy target part of the patient K1 is the lost left hand. The right hand R1 of the patient K1 is a counter part of the therapy target part. Hereinafter, of the therapy target part and the counter part of the therapy target part, an existing part is also referred to as a specific part. In a case of the patient K1, the right hand R1 is a specific part.

If the patient K1 puts the right hand R1 as a specific part into the box 50, the cheesecloth 30 is disposed between the specific part and the eyes of the patient K1. The patient K1 can visually recognize the right hand R1 put into the box 50 through the cheesecloth 30 and the transparent plate 40.

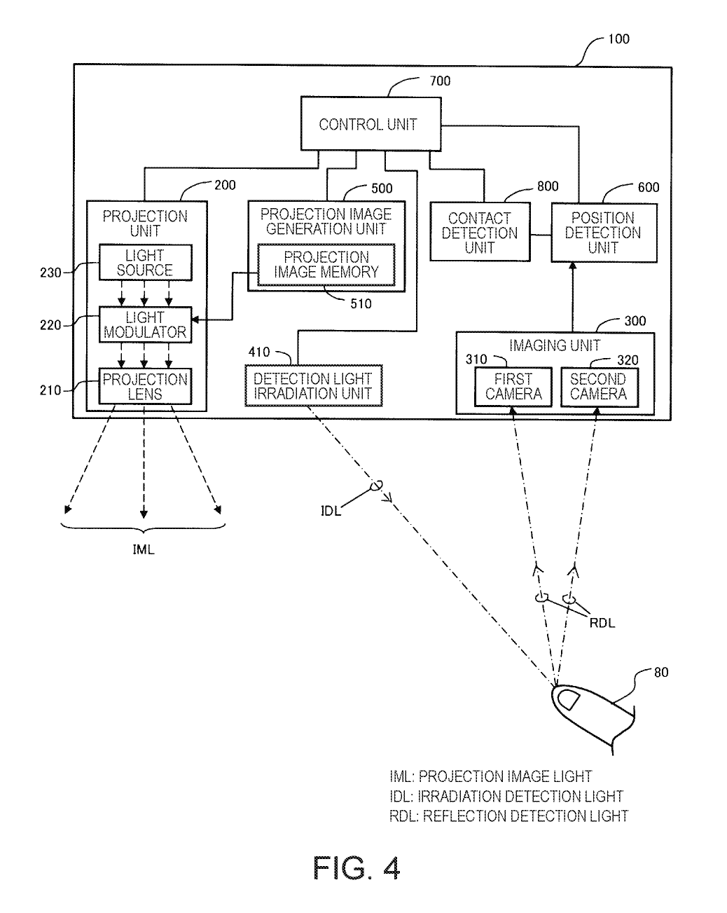

FIG. 4 is a block diagram illustrating an internal configuration of the projector 100. The projector 100 includes a projection unit 200, an imaging unit 300, a projection image generation unit 500, a position detection unit 600, a control unit 700 (computer), and a contact detection unit 800.

The control unit 700 controls the respective units of the projector 100. The control unit 700 determines the content of an instruction given by an indicator 80 on a dialogue screen D (which will be described later) on the basis of a three-dimensional position of the indicator 80 detected by the position detection unit 600, and contact of the indicator 80 detected by the contact detection unit 800. The indicator 80 is, for example, the finger of the patient K1 or an assistant. The control unit 700 instructs the projection image generation unit 500 to create or change a projection image according to the content of the instruction.

The projection image generation unit 500 includes a projection image memory 510 storing projection images, and has a function of generating a projection image projected by the projection unit 200. The projection image generation unit 500 further preferably functions as a keystone correction unit which corrects trapezoidal distortion of the projection screen.

The projection unit 200 has a function of projecting the projection image generated by the projection image generation unit 500. The projection unit 200 includes a projection lens 210, a light modulator 220, and a light source 230. The light modulator 220 modulates light from the light source 230 on the basis of projection image data provided from the projection image memory 510, so as to generate projection image light IML. The projection image light IML is typically color image light including visible light of three colors such as RGB, and is projected by the projection lens 210. As the light source 230, not only light source lamps such as an ultra-high pressure mercury lamp but also various light sources such as a light emitting diode or a laser diode may be employed. As the light modulator 220, a transmissive or reflective liquid crystal panel or a digital mirror device may be employed, and a plurality of light modulators 220 may be provided for respective color light beams.

The imaging unit 300 includes a first camera 310 and a second camera 320. The first camera 310 and the second camera 320 have a function of receiving light in a wavelength region including a wavelength of detected light, and performing imaging. Imaging in the first camera 310 and the second camera 320 is performed on both of a period in which irradiation detection light IDL is applied from a detection light irradiation unit 410 and a period in which the irradiation detection light IDL is not applied from the detection light irradiation unit 410.

At least one of the first camera 310 and the second camera 320 preferably has a function of performing imaging by using light including near infrared light and further a function of performing imaging by using light including visible light. In this way, the cameras can capture an image of a projected image, and the projection image generation unit 500 performs keystone correction by using the image. A method of performing keystone correction using one or more cameras is well known, and thus a description thereof will be omitted here.

The position detection unit 600 has a function of analyzing images captured by the first camera 310 and the second camera 320, and calculating three-dimensional position coordinates of a front end of the indicator 80 by using triangulation.

The contact detection unit 800 detects contact of the indicator 80 with the cheesecloth 30 on the basis of the three-dimensional position coordinates detected by the position detection unit 600.

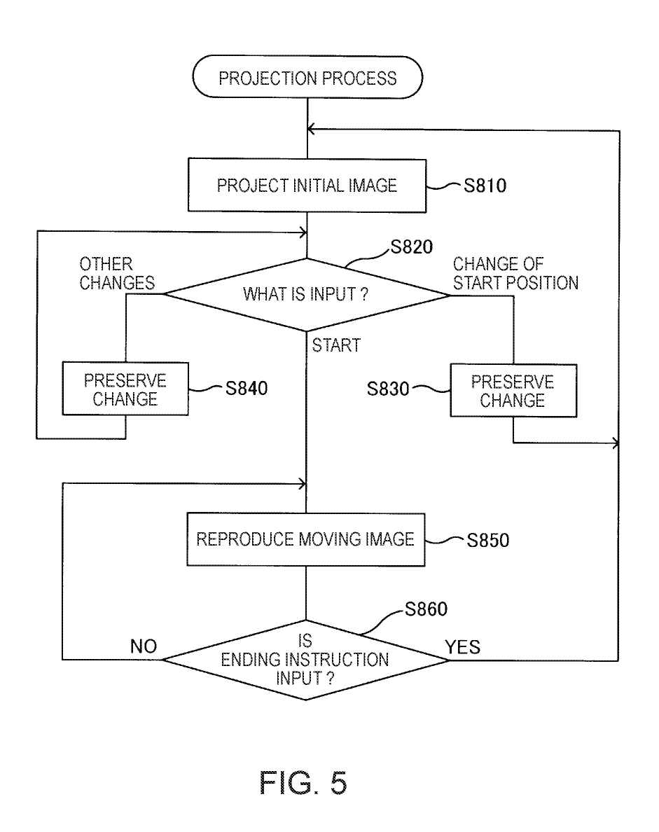

FIG. 5 is a flowchart illustrating a projection process. The projection process is performed by the control unit 700 executing a program stored in the control unit 700. The projection process includes a moving image generation process of generating moving images, and a display process of reproducing the generated moving images and displaying the moving images on a screen. The control unit 700 starts the projection process when power is supplied to the projector 100. First, an initial image SG is projected (step S810).

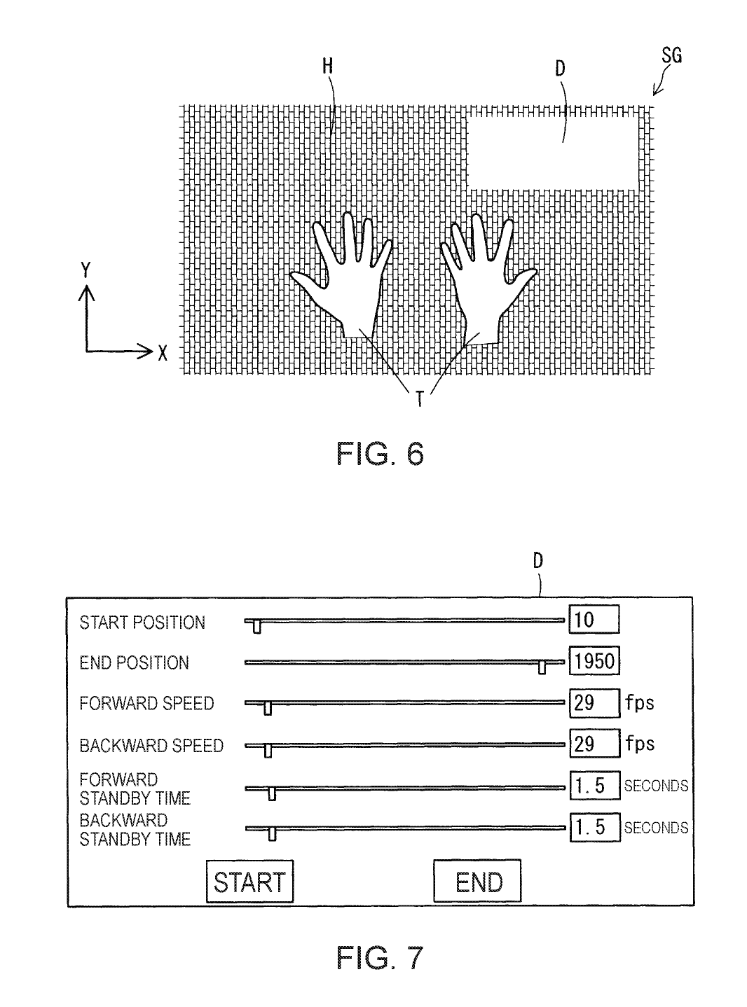

FIG. 6 illustrates the initial image SG. As illustrated in FIG. 6, the initial image SG includes a background H, the dialogue screen D, and images T in a first state. Nothing is displayed on the background H, and the background H is transmitted through the cheesecloth so as to be displayed, and thus is an image region of a blank and black portion. The images Tare images of the hands. The first state is a state in which the hands are opened. In contrast, a second state is a state in which the hands are closed. Both of the first and second states are states in which the backs of the hands face downward in the Z direction, and the palms face upward in the Z direction. Therefore, the images T in the initial image SG include images of the palms.

FIG. 7 is an enlarged view of the dialogue screen D. The dialogue screen D is a screen for setting and inputting reproduction conditions of the images T. Inputting to the dialogue screen D is performed by the patient K1 or an assistant.

As illustrated in FIG. 7, the dialogue screen D includes a start position, an end position, a forward speed, a backward speed, forward standby time, and backward standby time as setting items. Each of these setting items is provided with a slide bar and a display of a set value. The patient K1 traces the cheesecloth 30 with the finger so as to horizontally move the slide bar, and can thus change a set value of each setting item.

The start position is an item for setting an image used in the first state. As a numerical value is reduced, this corresponds to a state in which the hand is further opened. The end position is an item for setting an image used in the second state. As a numerical value is increased, this corresponds to a state in which the hand is further closed.

The forward speed is a speed at which an image changes in forward transition. The forward transition indicates transition from the first state to the second state. Conversely, transition from the second state to the first state is referred to as backward transition. A speed at which an image changes in the backward transition is the backward speed illustrated in FIG. 7.

A period of time is provided in which movement of the images T is stopped between an operation of the backward transition and an operation of the forward transition. Such stoppage of movement of the images T is referred to as forward standby, and a standby time is referred to as a forward standby time.

Similarly, a period of time is provided in which movement of the images T is stopped between an operation of the forward transition and an operation of the backward transition. Such stoppage of movement of the images T is referred to as backward standby, and a standby time is referred to as a backward standby time. Therefore, movement of the images T is performed in order of the forward transition, the backward standby, the backward transition, and the forward standby. The movement of the images T is repeatedly performed from the forward transition after the forward standby.

“Start” disposed under the setting items is display for starting an operation of the images T. “End” is display for finishing an operation of the images T.

After step S810, an instruction which is input via the dialogue screen D is determined (step S820). In a case where a start position is changed (change of start position in step S820), the changed start position is stored (step S830), and the flow returns to step S810. The changed start position is reflected in second step S810.

In a case where set values other than the start position are changed (other changes in step S820), changed set values are stored (step S840), and the flow returns to step S820. In a case where a starting instruction is input (start in step S820), moving images in which the images T change are reproduced (step S850).

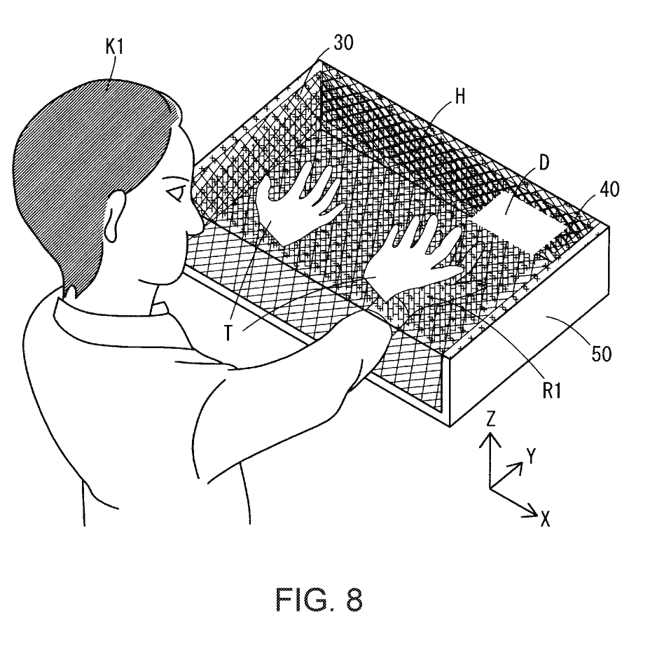

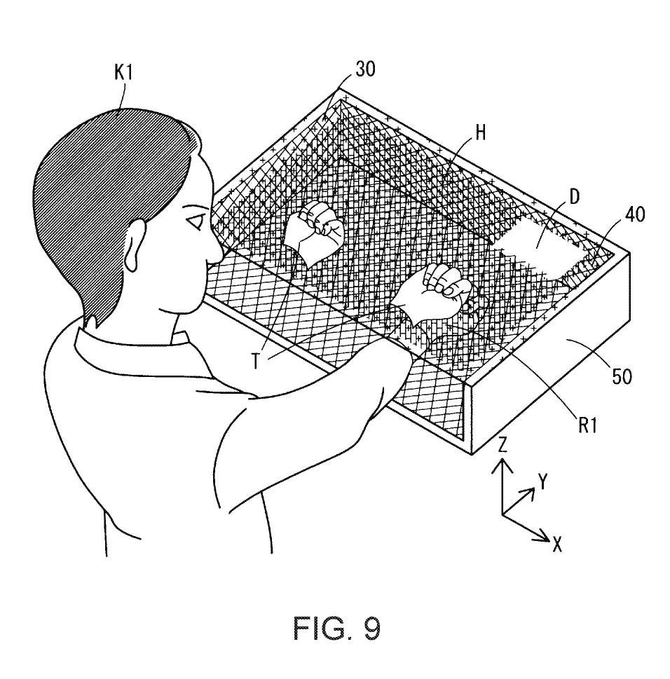

FIG. 8 is a diagram illustrating the first state during reproduction of the moving images. FIG. 9 is a diagram illustrating the second state during reproduction of the moving images. The patient K1 visually recognizes the images T and the right hand R1 and opens or closes the right hand R1 in accordance with movement of the images T. The patient K1 imagines that the lost left hand is closed or opened in accordance with the movement of the images T.

The moving images are continuously reproduced if an ending instruction is not input (NO in step S860). In a case where the ending instruction is input (YES in step S860), the flow returns to step S810. The projection process is continuously performed until the supply of power to the projector 100 is stopped.

According to the present embodiment, at least the following effects can be achieved.

(1) The cheesecloth 30 as a screen member is black, and the background H projected by the projector 100 is transmitted through the cheesecloth 30 so as to be displayed. Therefore, the patient K1 viewing the images T visually recognizes the background H in black and thus is absorbed in the image. Therefore, an illusion is enhanced, and thus it is possible to expect a therapeutic effect to be improved.

(2) The patient K1 disposes the hand which is not lost directly under the image T, and can visually recognize the hand thereof emulating the movement of the image T. Therefore, the patient K1 can recognize that the movement is emulated.

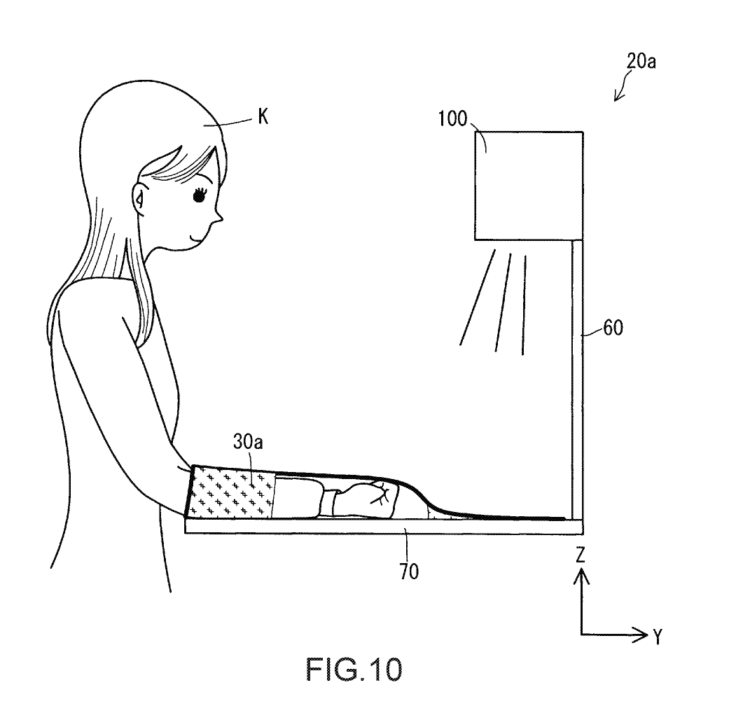

FIG. 10 is a side view of a medical apparatus 20a in Embodiment 2. The medical apparatus 20a includes a cloth 30a, a column 60, a stand 70, and a projector 100. The column 60 and the projector 100 are the same as those in Embodiment 1, and thus a description thereof will be omitted.

The stand 70 supports the column 60 and allows the cloth 30a, and the forearms and the hands of a patient K to be placed thereon. The cloth 30a is made of a material which is black, soft, and has light blocking property. FIG. 10 illustrates apart of the cloth 30a which is virtually notched.

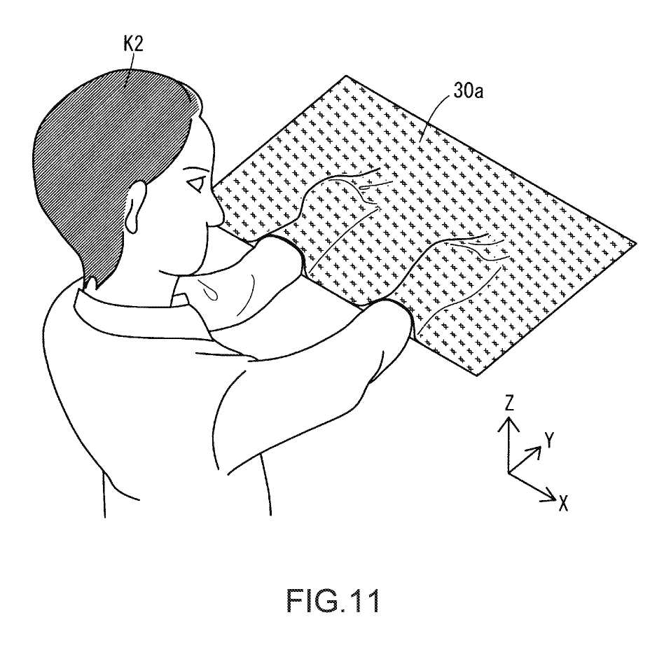

FIG. 11 illustrates a state in which a patient K2 puts both hands thereof between the cloth 30a and the stand 70. The patient K2 has paralytic symptoms in the left hand thereof. A therapy target part of the patient K2 is the left hand suffering from the paralytic symptoms. The right hand of the patient K2 is a counter part of the therapy target part. Specific parts of the patient K2 are the right hand and the left hand.

If the patient K2 puts the right hand and the left hand between the cloth 30a and the stand 70, the cloth 30a as a screen member is disposed between the specific parts and the eyes of the patient K2. The cloth 30a is soft, and thus rises up along the shapes of the hands as illustrated in FIG. 11.

Also in Embodiment 2, the same projection process as in Embodiment 1 is performed. Hereinafter, the first state and the second state in Embodiment 2 will be described.

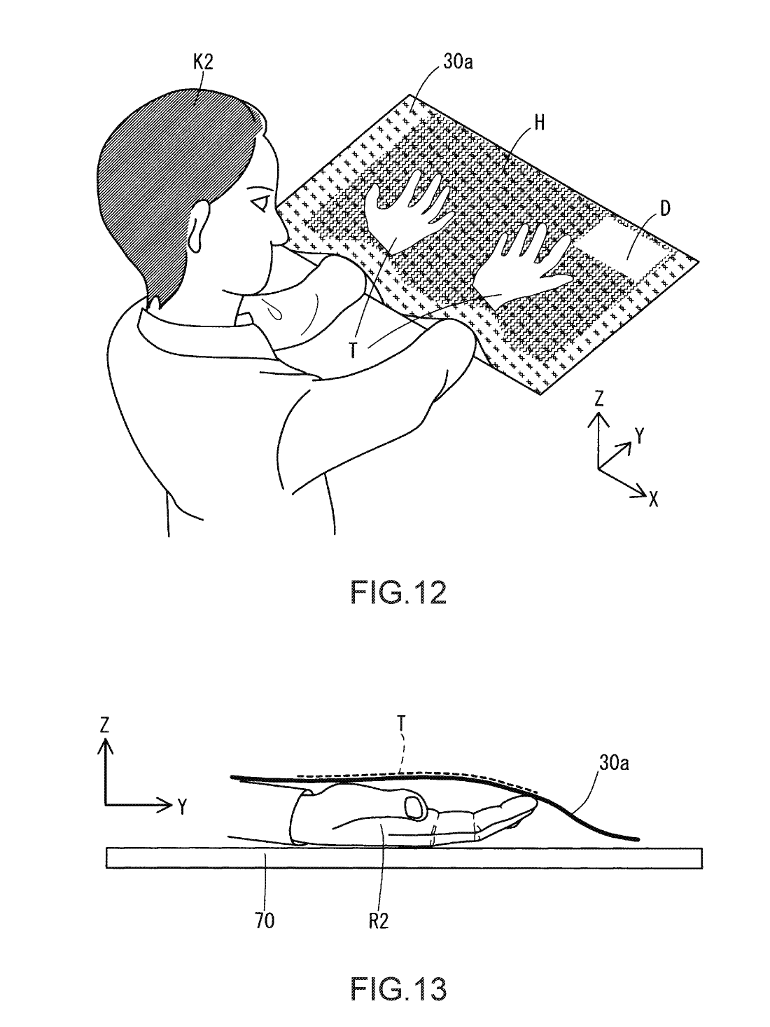

FIG. 12 is a diagram illustrating the first state during reproduction of moving images (step S850). FIG. 13 is a sectional view in the first state. The patient K2 disposes the right hand R2 and the left hand (not illustrated) at XY direction positions at which the images T are projected.

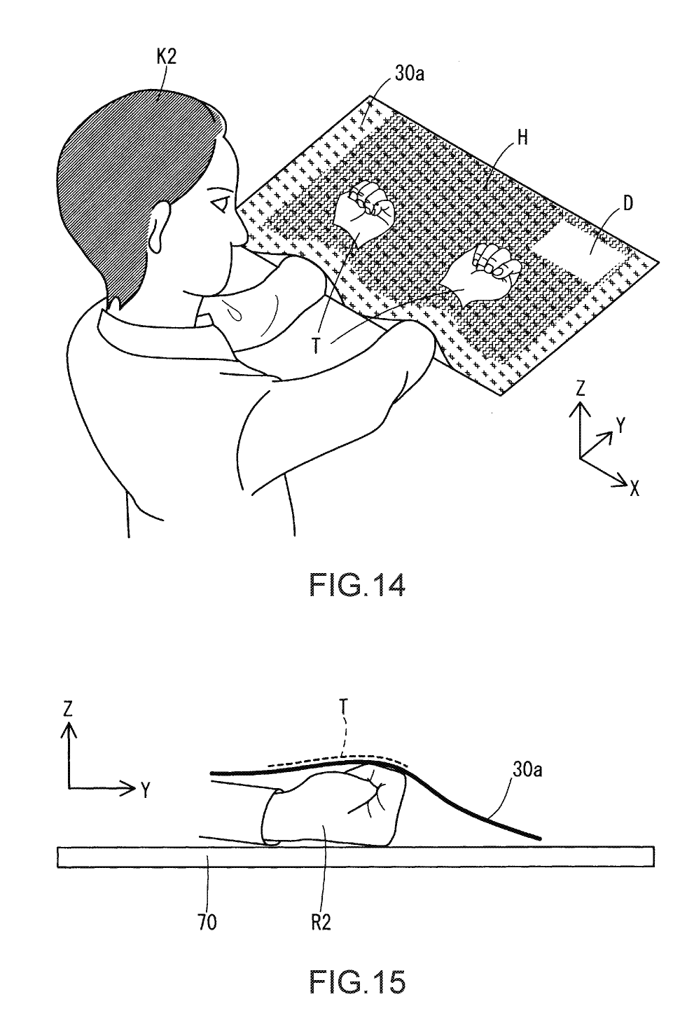

FIG. 14 is a diagram illustrating the second state during reproduction of the moving images. FIG. 15 is a sectional view in the second state.

In both of the first state and the second state, parts onto which the images T are projected rise up by the right hand R2 and the left hand, and thus the images T are viewed in a stereoscopic manner. Thus, an immersion feeling is enhanced, and thus it is possible to expect a therapeutic effect to be improved.

The invention is not limited to the embodiments, Examples, and modification examples of the present specification, and may be realized in various configurations within the scope without departing from the spirit thereof. For example, the technical features in the embodiments, Examples, and modification examples corresponding to the technical features disclosed in Summary of the Invention may be replaced or combined with each other as appropriate in order to solve some or all of the above-described problems or in order to achieve some or all of the above-described effects. The technical features may be omitted as appropriate as long as the technical features are not described as being essential. This is as follows, for example.

A definition of “black” is not limited to the above definition, and any color may be defined as long as the color provides an immersion feeling to a patient, and thus improvement of a therapeutic effect can be expected.

There may be various methods of displaying moving images in which a therapy target part repeatedly moves and a black background. For example, a see-through display may be used as a configuration of combining a screen member with a display device. Since the see-through display allows an opposite side to be viewed therethrough, therapy can be performed in the same manner as in Embodiment 1. As the screen member, a half mirror, a light controllable glass, a liquid crystal panel, and the like may be used.

In Embodiment 1, if a rigid member is used instead of the cheesecloth, the transparent plate may not be used. The rigid member is preferably black and allows an opposite side to be viewed therethrough in the same manner as the cheesecloth.

In a case where moving images are displayed through projection using a projector as in Embodiments 1 and 2, the projector may project only parts corresponding to the moving images and may not project a background.

The medical apparatus may be used for therapy of a patient who has lost both hands and thus feels phantom limb pain of both hands. In this case, the screen member may not be transparent, and a space for disposing the hands and the forearms may not be provided.

In Embodiment 2, the cloth as a screen member may be translucent.

A moving image for a normal part of a patient may not be displayed. For example, in a case of Embodiment 1, a moving image for the right hand may not be projected. In this case, the patient only may imagine that the lost left hand moves in accordance with the moving image, and may not move the right hand. In this case, the screen member may not be translucent. Even in a case where a moving image for a normal part is displayed, the screen member may not be translucent.

The setting items using the dialogue screen may be changed. For example, a size of the hand, a color of the hand, a distance between the right hand and the left hand, a position of the hand, and an angle of the hand may be added. The position of the hand is a two-dimensional position, that is, a position in the X-Y direction. The angle of the hand is a position in a rotation direction when the hand is rotated with respect to a longitudinal direction of the forearm. If an angle of the hand is changed, for example, the palm or the back of the hand faces upward in the Z direction.

A dialogue screen may not be provided by using a projector not having an interactive function. In this case, there may be a configuration in which a set value is changed by using a physical switch provided in the projector, and there may be a configuration in which a PC as an external apparatus is connected to the projector, and a set value is changed via the PC.

The medical apparatus of Embodiment 1 may be used for rehabilitation of paralysis.

The medical apparatus of Embodiment 2 may be used for therapy of phantom limb pain.

A therapy target part may not be the hand. For example, the medical apparatus may be used for therapy performed by imagining that an elbow joint, a shoulder joint, a hip joint, or an ankle moves.

In the respective embodiments, both of generation of moving images and projection of the moving images are performed as the projection process, but generation and projection of the moving images may be performed separately. The projection process is performed according to the program stored in the control unit 700 of the projector, but an external computer connected to the projector may generate moving images, and the projector may project the moving images.

The entire disclosure of Japanese Patent Application No. 2015-159743 filed Aug. 13, 2015 is expressly incorporated by reference herein.

What is claimed is:

1. A medical apparatus comprising:
a computer that displays a moving image in which a therapy target part moves on a screen member,
wherein the screen member is black.

2. The medical apparatus according to claim 1,
wherein at least a background portion is black in the screen member.

3. The medical apparatus according to claim 1,
wherein the computer projects the moving image having a background which is transmitted and displayed, onto the screen member.

4. The medical apparatus according to claim 1,
wherein the screen member is disposed between a specific part which is at least one of the therapy target part and a counter part of the therapy target part, and the eyes of a patient.

5. The medical apparatus according to claim 4,
wherein the screen member is translucent.

6. The medical apparatus according to claim 4,
wherein the screen member has a shape rising up along the specific part during display of the moving image, and
wherein the moving image is displayed on the rising-up part.

7. A storage medium storing a program causing a computer to execute:
generating a moving image in which a therapy target part moves, a background of the therapy target part being black.
